(19) United States
(12) Patent Application Publication
(10) Pub. No.: US 2004/0073185 A1
(43) Pub. Date: Apr. 15, 2004

(54) Absorbent article

(76) Inventors: Yuzo Ichiura, Osaka (JP); Masaki Nakakado, Osaka (JP); Syuhei Kurata, Kyoto (JP); Natsumi Murata, Osaka (JP)

Correspondence Address: ESCHWEILER & ASSOCIATES, LLC, NATIONAL CITY BANK BUILDING, 629 EUCLID AVE., SUITE 1210, CLEVELAND, OH, 44114, US

(21) Appl. No.: 10/674,741

(22) Filed: Sep. 30, 2003

(30) Foreign Application Priority Data
Oct. 9, 2002 (JP) 2002-295916

(51) Int. Cl. A61F 13/15; A61F 13/20

(52) U.S. Cl. 604/385.201; 604/385.04

(57) ABSTRACT

An absorbent article, comprising a main part, the main part including a liquid-permeable surface sheet, a liquid-impermeable anti leak sheet, and a liquid-holding absorbent provided between the surface sheet and the anti leak sheet. The main part is folded into a plurality of layers, and portions of the folded main part that are overlapping with one another along opposite side edges of the main part are bonded together.

### BACKGROUND OF THE INVENTION

1. Field of the Invention

The present invention relates to an absorbent article that is put on the inside of the user's underwear in a narrow crotch area, such as a sanitary napkin, an incontinence pad, a sanitary pad for vaginal discharges, or the like.

2. Description of the Related Art

Typically, an absorbent article such as a sanitary napkin at least includes an elongate main part, including a liquid-permeable surface sheet, a liquid-impermeable anti leak sheet, and a liquid-holding absorbent provided between the surface sheet and the anti leak sheet. The absorbent article is placed on a packaging sheet that is large enough to extend beyond the front, back, first and second edges of the main part, with the anti leak sheet side of the absorbent article facing the packaging sheet. The absorbent article is folded in three in the longitudinal direction. Specifically, the front portion of the main part is folded, together with the front portion of the packaging sheet, onto the middle portion of the main part, and the rear portion of the main part is folded, together with the rear portion of the packaging sheet, onto the already-folded front portion of the packaging sheet, thereby obtaining an individually-packaged, three-fold absorbent article (see, for example, Japanese Laid-Open Patent Publication Nos. 9-10257 and 9-327478).

Since the absorbent article is individually packaged with the packaging sheet, the folded absorbent article (particularly the surface sheet thereof, which is to be in contact with the user's skin) can be kept unexposed until use. However, before using the absorbent article, the user needs to open the packaging sheet, and the packaging sheet needs to be disposed of as a waste.

Thus, the provision of the packaging sheet for individually packaging the absorbent article not only consumes natural resources but also imposes an additional cost for the disposal thereof.

### SUMMARY OF THE INVENTION

It is an object of the present invention to provide an absorbent article that is kept folded while keeping its surface sheet unexposed until use, without using a packaging sheet.

An absorbent article of the present invention at least includes an elongate main part, the main part including a liquid-permeable surface sheet, a liquid-impermeable anti leak sheet, and a liquid-holding absorbent provided between the surface sheet and the anti leak sheet, wherein the main part is folded into a plurality of layers, and portions of the surface sheet and the anti leak sheet of the folded main part that are overlapping with one another along opposite side edges of the main part are bonded together.

Thus, the main part is folded into a plurality of layers, and portions of the folded main part that are overlapping with one another along the opposite side edges of the main part are bonded together, whereby the absorbent article can be kept folded while keeping the surface sheet, which is to be in contact with the user's skin, unexposed.

As a result, it is no longer necessary to provide a packaging sheet for individually packaging the absorbent article, thereby conserving natural resources and saving the cost for the disposal thereof.

In the present invention, a wing portion may extend from each side edge of the surface sheet and the anti leak sheet of the main part. In such a case, as the wing portions are folded back onto the outer surface of the user's underwear, a portion of each side edge portion of the main part, which is used as a bonding portion, is also folded back onto the outer surface of the user's underwear. Thus, it is possible to prevent a bonding portion from contacting and irritating the user's skin.

In the present invention, each wing portion may be folded onto the surface sheet with an adhesive being applied on an outer surface thereof, and a release liner may be attached to the wing portions so as to extend between the adhesive application areas of the wing portions. Thus, the wing portions can be kept folded onto the surface sheet. As the release liner is peeled off and the wing portions are folded back onto the outer surface of the user's underwear, the wing portions can be attached to the outer surface of the user's underwear via the adhesive application areas. Thus, the absorbent article can be fixed on the user's underwear. In the present invention, an adhesive may be applied to at least a portion of the anti leak sheet, with a release liner being attached to the adhesive application area of the anti leak sheet, while the main part is folded. Then, when using the absorbent article, the absorbent article can be fixed on the user's underwear.

### BRIEF DESCRIPTION OF THE DRAWINGS

FIG. 1 is a perspective view illustrating a sanitary napkin as an absorbent article of the present invention according to one embodiment of the present invention.

FIG. 2 is a cross-sectional view illustrating the sanitary napkin of FIG. 1 taken along line X-X in FIG. 1.

FIG. 3 is a perspective view illustrating the sanitary napkin of FIG. 1 after unfolding the sanitary napkin by peeling off the bonded portions.

FIG. 4 is a perspective view illustrating the sanitary napkin of FIG. 1 immediately before it is put on the user's underwear.

FIG. 5A is a plan view illustrating a sanitary napkin as an absorbent article of the present invention according to an alternative embodiment of the present invention, FIG. 5B is a plan view illustrating the sanitary napkin of FIG. 5A after unfolding the sanitary napkin by peeling off the bonded portions, and FIG. 5C is a cross-sectional view taken along line Y-Y in FIG. 5A.

FIG. 6A is a plan view illustrating a sanitary napkin as an absorbent article of the present invention according to another alternative embodiment of the present invention, FIG. 6B is a plan view illustrating the sanitary napkin of FIG. 6A after unfolding the sanitary napkin by peeling off the bonded portions, and FIG. 6C is a cross-sectional view taken along line Y-Y in FIG. 6A.

### DESCRIPTION OF THE PREFERRED EMBODIMENTS

Preferred embodiments of the present invention will now be described with reference to the drawings.

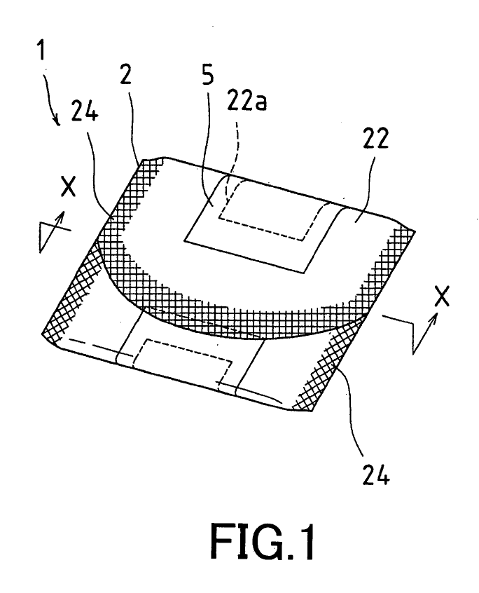

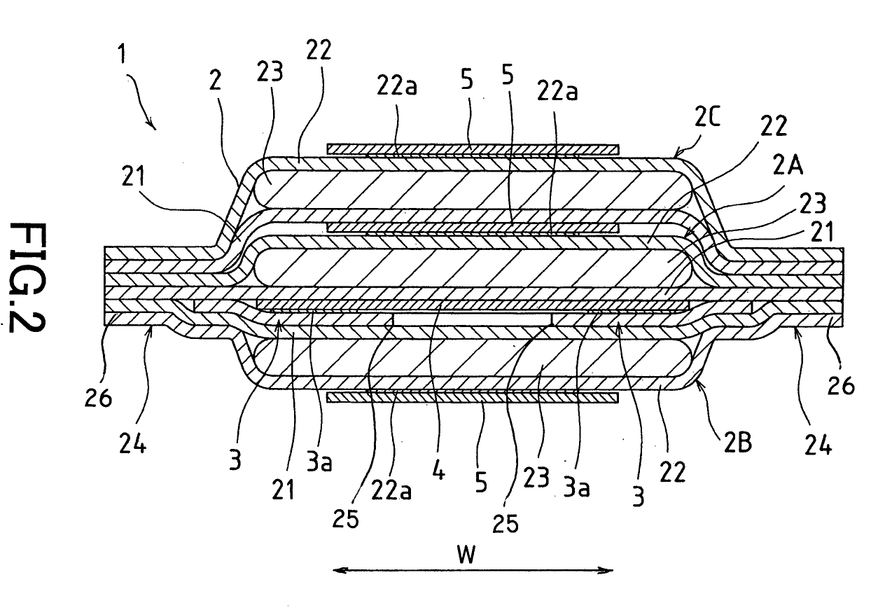

FIG. 1 and FIG. 2 illustrate a sanitary napkin 1 as an absorbent article of the present invention according to one embodiment of the present invention.

The sanitary napkin 1 includes an elongate main part 2, and a pair of wing portions 3 having a generally trapezoidal shape that are integral respectively with first and second side edge portions 24 of the main part 2 in a middle portion 2B, which is located generally in the middle of the main part 2. The first side edge opposes to the second side edge. The main part 2 includes a liquid-permeable surface sheet 21, a liquid-impermeable anti leak sheet 22, and a liquid-holding absorbent 23 provided between the surface sheet 21 and the anti leak sheet 22. The wing portions 3 extend respectively from the first and second side edge portions 24 of the main part 2 (more specifically, from the first and second side edge portions of the surface sheet 21).

Each wing portion 3 is normally folded onto the surface sheet 21, and includes an adhesive application area 3a where an adhesive is applied on the surface of the wing portion 3. Moreover, a release liner 4 is attached to the adhesive application areas 3a of the first and second wing portions 3 so as to extend between the adhesive application areas 3a, whereby the free end 25 of each wing portion 3 is held in contact with the surface sheet 21 so as not to extend in the width direction W beyond the first or second side edge portion 24 of the main part 2.

Moreover, the anti leak sheet 22 includes an adhesive application area 22a where an adhesive is applied on the surface of the anti leak sheet 22 in the longitudinal direction L, and a release liner 5 is attached to the adhesive application area 22a.

Note that the wing portions 3 may be connected to the anti leak sheet 22, instead of connecting them to the surface sheet 21. For example, in a case where portions 26 of the anti leak sheet 22 are not covered by the surface sheet 21 at the first and second side edge portions 24 of the main part 2, the wing portions 3 may be connected to the portions 26.

The sanitary napkin 1 is folded in three with the wing portions 3 being folded onto the surface sheet 21 of the main part 2. Specifically, a front portion 2A of the main part 2 is folded onto the middle portion 2B, with the surface sheet 21 of the front portion 2A being in contact with the surface sheet 21 of the middle portion 2B, and then a rear portion 2C is folded onto the already-folded front portion 2A, with the surface sheet 21 of the rear portion 2C being in contact with the anti leak sheet 22 of the front portion 2A, thereby obtaining a three-fold structure. Then, portions of the surface sheet 21 and the anti leak sheet 22 of the main part 2 that are overlapping with one another along the first and second side edge portions 24 of the sanitary napkin 1, which has been folded into a three-fold structure, are bonded together by using thermal fusion bonding, ultra sonic fusion bonding, an adhesive, or the like. Thus, the surface sheet 21, which is to be in contact with the user's skin, is kept unexposed.

The bonding strength between the surface sheet 21 of the front portion 2A and the surface sheet 21 of the middle portion 2B is set to be smaller than that between the wing portions 3 and the surface sheet 21. Moreover, the bonding strength between the surface sheet 21 of the rear portion 2C and the anti leak sheet 22 of the front portion 2A is set to be smaller than that between the wing portions 3 and the surface sheet 21. Furthermore, in a case where the surface sheet 21 of the front portion 2A and the wing portions 3 are bonded to each other, the bonding strength therebetween is set to be smaller than that between the wing portions 3 and the surface sheet 21 (or the anti leak sheet 22) of the middle portion 2B.

As a result, the sanitary napkin 1 is folded into a three-fold structure and is kept in the three-fold structure by bonding along the first and second side edge portions 24, so that the surface sheet 21, which is to be in contact with the user's skin, is kept unexposed. Therefore, it is no longer necessary to provide a packaging sheet, thereby conserving natural resources and saving the cost for the disposal thereof.

Next, how to use the sanitary napkin 1 as described above will be described.

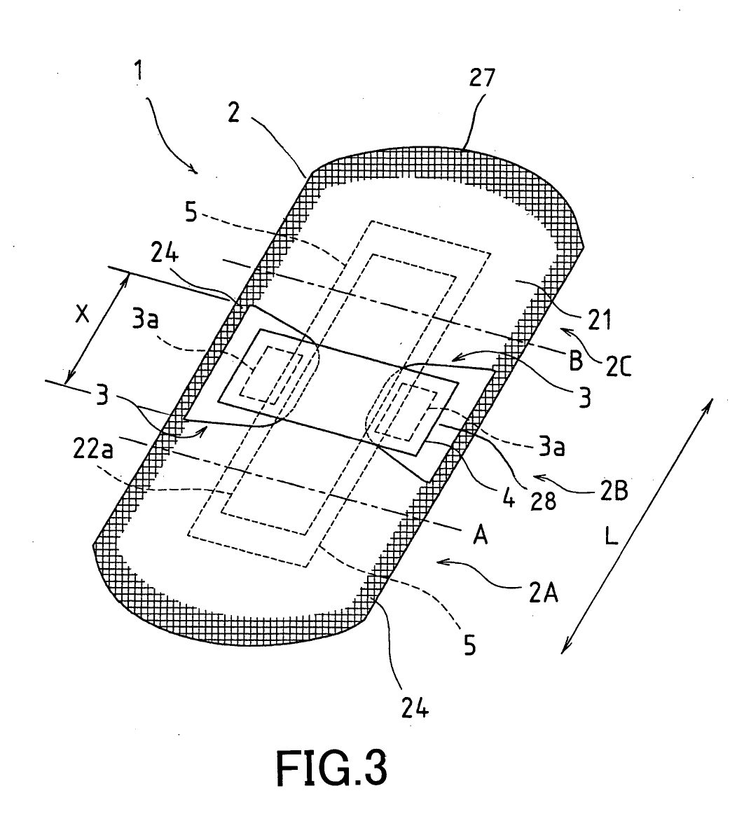

First, as illustrated in FIG. 3, the user takes the tip 27 of the rear portion 2C of the main part 2 of the three-fold sanitary napkin 1, and unfolds the rear portion 2C, thereby peeling off the first and second side edge portions of the surface sheet 21 and the anti leak sheet 22 of the rear portion 2C from those of the front portion 2A so that the rear portion 2C forms a continuous flat surface with the middle portion 2B. Similarly, the user takes the tip of the front portion 2A of the main part 2, and unfolds front portion 2A, thereby peeling off the side edge portions 24 of the surface sheet 21 and the anti leak sheet 22 of the front portion 2A from those of the middle portion 2B so that the front portion 2A forms a continuous flat surface with the middle portion 2B (see FIG. 3).

Then, the release liner 5 attached to the anti leak sheet 22 is peeled off from the adhesive application area 22a, and the adhesive application area 22a is attached to the crotch portion of the inner surface of the user's underwear. Then, the release liner 4 is peeled off from the adhesive application areas 3a of the wing portions 3, and the wing portions 3 are folded back around the crotch portion of the underwear to be attached to the outer surface thereof. Thus, the sanitary napkin 1 can be fixed on the underwear.

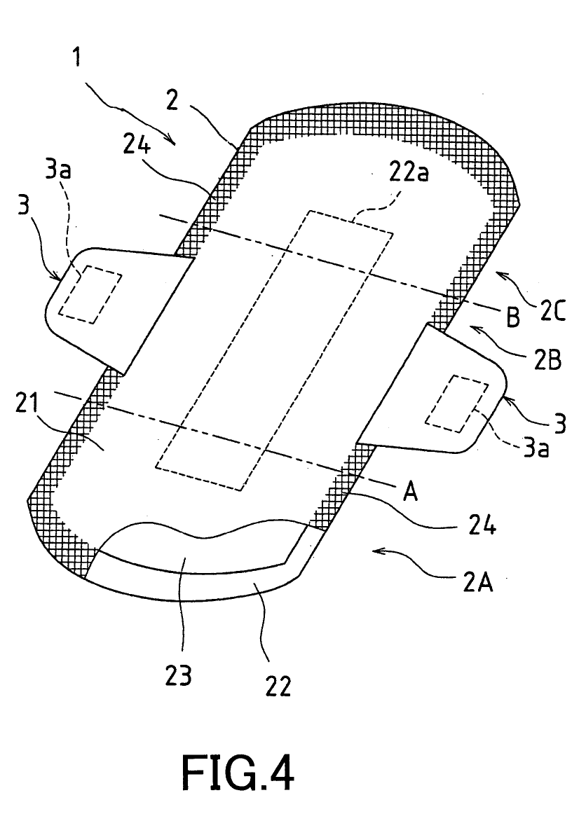

FIG. 4 illustrates a plan view of the sanitary napkin 1 just prior to being applied to the user's underwear. As the wing portions 3 are folded back outwardly, a portion of each side edge portion of the surface sheet 21 and the anti leak sheet 22 of the main part 2, i.e., each side edge portion of the middle portion 2B, which is used as a bonding portion, is also folded back onto the outer surface of the user's underwear, together with the wing portions 3, thus preventing a bonding portion from contacting and irritating the user's skin.

While the present embodiment is directed to the sanitary napkin 1 including the wing portions 3, the present invention is not limited to the sanitary napkin 1, and the wing portions 3 may be optional under certain circumstances.

While the wing portions 3 are provided in the middle portion 2B of the main part 2 in the present embodiment, the position where the wing portions 3 are provided is not limited to any particular position, and they may alternatively be provided in the front portion 2A or the rear portion 2C, for example, as long as the sanitary napkin 1 can be fixed on the user's underwear.

While the sanitary napkin 1 is folded into a three-fold structure in the present embodiment, it may alternatively be folded in two or four, for example.

While the wing portions 3 are provided in the middle portion 2B of the main part 2 in the present embodiment so that when the wing portions 3 are folded back, the side edge portions 24 of the middle portion 2B, which are used as bonding portions, are folded back onto the outer surface of the user's underwear, it may be preferred that the side edge portions 24 of the main part 2 are entirely folded back onto the outer surface of the underwear by, for example, increasing the width X of the foot portion 28 of the wing portions 3.

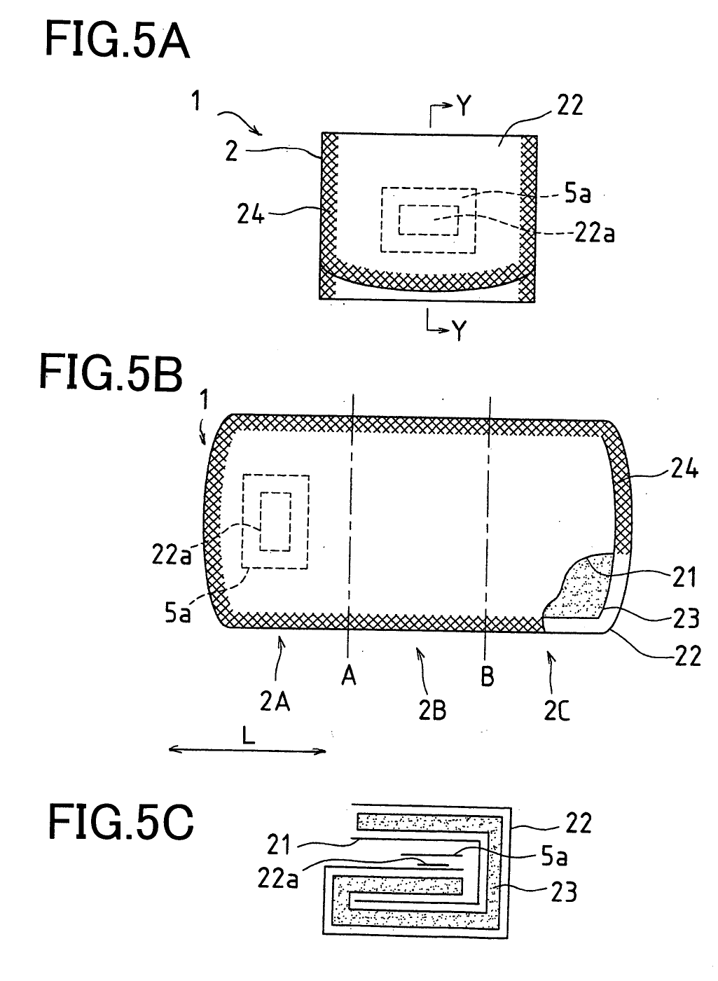

While an adhesive is applied on the surface of the anti leak sheet 22 to provide the adhesive application area 22a in the present embodiment, the adhesive application area 22a may be optional. An alternative embodiment of the present invention will now be described with reference to FIG. 5A to FIG. 5C. As illustrated in FIG. 5A, the sanitary napkin 1 of the present embodiment includes the elongate main part 2, including the liquid-permeable surface sheet 21, the liquid-impermeable anti leak sheet 22, and the liquid-holding absorbent 23 provided between the surface sheet 21 and the anti leak sheet 22. Moreover, the anti leak sheet 22 includes the adhesive application area 22a, where an adhesive is applied, near one end of the anti leak sheet 22 in the longitudinal direction L, and a release liner 5a is attached to the adhesive application area 22a. The main part 2 is folded in three so that the release liner 5a is kept unexposed. Specifically, referring to FIG. 5B, an end portion 2A of the main part 2 is folded along line A onto a middle portion 2B so that the surface sheet of the end portion 2A faces the surface sheet of the middle portion 2B, and an end portion 2C is folded along line B, thereby forming a three-fold sanitary napkin. As a result, the main part 2 is folded so that the adhesive application area 22a and the release liner 5a are kept unexposed, with the surface sheet of the end portion 2C facing the release liner 5a, as illustrated in FIG. 5C. Then, portions of the surface sheet 21 and the anti leak sheet 22 of the main part 2 that are overlapping with one another along the first and second side edge portions 24 of the sanitary napkin 1, which has been folded into a three-fold structure, are bonded together by using thermal fusion bonding, ultrasonic fusion bonding, an adhesive, or the like. Thus, the surface sheet 21, which is to be in contact with the user's skin, is kept unexposed. While the adhesive application area 22a is provided near one of the main part 2 in the present embodiment, the present invention is not limited to this as long as it is provided near at least one end of the anti leak sheet 22. For example, adhesive application areas may be provided near both end portions 2A and 2C of the main part 2. Moreover, the release liner may be partially protruding out of the main part 2 as it is folded. Furthermore, the main part 2 may alternatively include wing portions as described above.

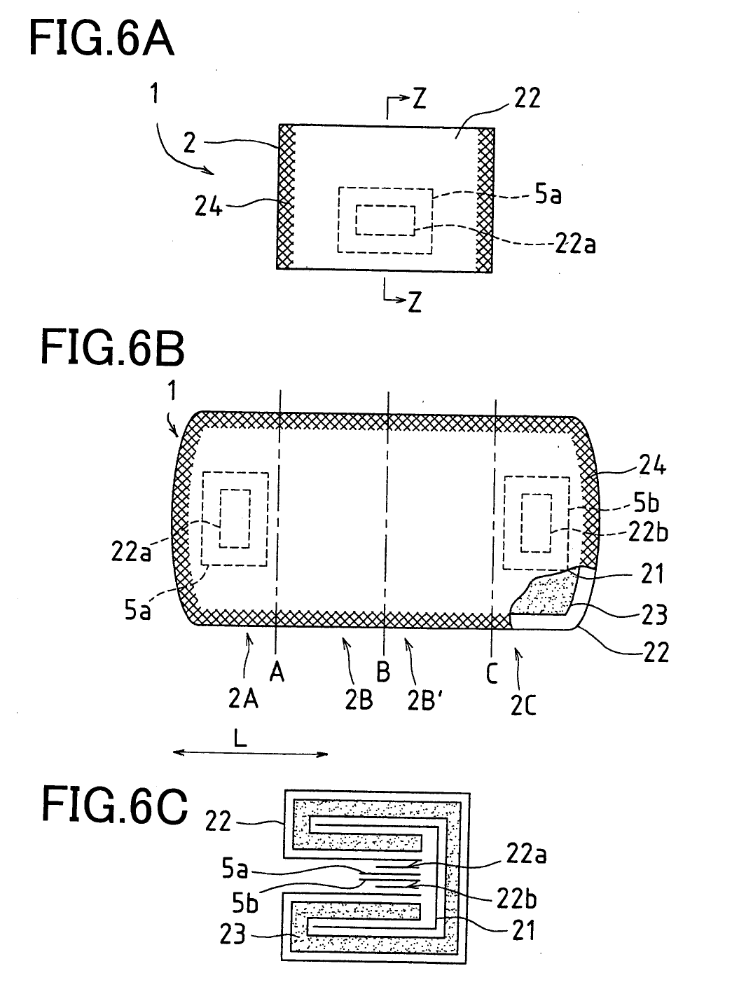

Next, another alternative embodiment of the present invention will be described with reference to FIG. 6A to FIG. 6C. As illustrated in FIG. 6A to FIG. 6C, the sanitary napkin 1 of the present embodiment includes the elongate main part 2, including the liquid-permeable surface sheet 21, the liquid-impermeable anti leak sheet 22, and the liquid-holding absorbent 23 provided between the surface sheet 21 and the anti leak sheet 22. Moreover, the anti leak sheet 22 includes adhesive application areas 22a and 22b, where an adhesive is applied, near opposite ends of the anti leak sheet 22 in the longitudinal direction L, and release liners 5a and 5b are attached to the adhesive application areas 22a and 22b, respectively. The sanitary napkin 1 is folded in four so that the release liners 5a and 5b are kept unexposed. Specifically, referring to FIG. 6B, an end portion 2A is folded along line A onto a middle portion 2B so that the surface sheet 21 of the end portion 2A faces the surface sheet 21 of the middle portion 2B. Similarly, an end portion 2C is folded along line C onto a middle portion 2B′ so that the surface sheet 21 of the end portion 2C faces the surface sheet 21 of the middle portion 2B′. The thus-folded main part 2 is further folded along line B, thereby forming a four-fold sanitary napkin. As a result, the main part 2 is folded so that the adhesive application areas 22a and 22b and the release liners 5a and 5b are kept unexposed, as illustrated in FIG. 6C. Then, portions of the surface sheet 21 and the anti leak sheet 22 of the main part 2 that are overlapping with one another along the first and second side edge portions 24 of the sanitary napkin 1, which has been folded into a four-fold structure, are bonded together by using thermal fusion bonding, ultrasonic fusion bonding, an adhesive, or the like. Thus, the surface sheet 21, which is to be in contact with the user's skin, is kept unexposed.

While the adhesive application areas 22a and 22b are provided near opposite ends of the main part 2 in the present embodiment, the present invention is not limited to this as long as it is provided near at least one of the anti leak sheet 22. For example, an adhesive application area may be provided near only one end, such as end portion 2A of the main part 2. Moreover, the release liners may be partially protruding out of the main part 2 as it is folded. In such a case, it is preferred that the protruding portions of the release liners are folded outwardly so that they do not add to the bulkiness of the folded sanitary napkin 1. Furthermore, the main part 2 may alternatively include wing portions as described above. Moreover, a single release liner may alternatively be provided, while being folded, between the adhesive application areas 22a and 22b. In such a case, it is preferred that at least one of the adhesive application areas 22a and 22b is formed in, for example, a striped pattern, a cross pattern, a dotted pattern, or the like, so that the end portion 2A or 2C is not entirely an adhesive application area, whereby the air trapped inside as the sanitary napkin 1 is folded can escape out of the folded sanitary napkin 1. Moreover, in such a case, it is preferred that the release liners are partially protruding out of the main part 2 so that the user can take the protruding portion to unfold the sanitary napkin 1 for use. It is preferred that the protruding portions of the release liners are folded outwardly so that they do not add to the bulkiness of the folded sanitary napkin 1.

Thus, according to the present invention, it is possible to keep an absorbent article such as a sanitary napkin folded until use without using a packaging sheet. Therefore, it is no longer necessary to provide a packaging sheet for individually packaging the absorbent article, thereby conserving natural resources and saving the cost for the disposal thereof.

What is claimed is:

1. An absorbent article, comprising a main part, the main part including a liquid-permeable surface sheet, a liquid-impermeable anti leak sheet, and a liquid-holding absorbent provided between the surface sheet and the anti leak sheet, wherein the main part is folded into a plurality of layers, and portions of the folded main part that are overlapping with one another along opposite side edges of the main part are bonded together.

2. An absorbent article according to claim 1, wherein a wing portion extends from each side edge of the main part.

3. An absorbent article according to claim 2, wherein each wing portion is folded onto the surface sheet with an adhesive being applied on an outer surface thereof, therein defining an adhesive application area, and a release liner is attached to the wing portions so as to extend between the adhesive application areas of the wing portions.

4. An absorbent article according to claim 1, wherein an adhesive is applied to at least a portion of anti leak sheet, therein defining an adhesive application area, with a release liner being attached to the adhesive application area of the anti leak sheet.

5. An absorbent article according to claim 2, wherein an adhesive is applied to at least a portion of anti leak sheet, therein defining an adhesive application area, with a release liner being attached to the adhesive application area of the anti leak sheet.

6. An absorbent article according to claim 1, wherein the main part is folded in two or more.

7. An absorbent article according to claim 2, wherein the main part is folded in two or more.

8. An absorbent article according to claim 3, wherein the main part is folded in two or more.

9. An absorbent article according to claim 4, wherein the main part is folded in two or more.

10. An absorbent article according to claim 1, wherein the bonding is done by using at least one of thermal fusion bonding, ultrasonic fusion bonding and an adhesive.

11. An absorbent article according to claim 2, wherein the bonding is done by using at least one of thermal fusion bonding, ultrasonic fusion bonding and an adhesive.

12. An absorbent article according to claim 3, wherein the bonding is done by using at least one of thermal fusion bonding, ultrasonic fusion bonding and an adhesive.

13. An absorbent article according to claim 4, wherein the bonding is done by using at least one of thermal fusion bonding, ultrasonic fusion bonding and an adhesive.

14. An absorbent article according to claim 5, wherein the bonding is done by using at least one of thermal fusion bonding, ultrasonic fusion bonding and an adhesive.

15. An absorbent article according to claim 2, wherein bonding strength between the surface sheet and the surface sheet is set to be smaller than that between the wing portions and the surface sheet.

16. An absorbent article according to claim 3, wherein bonding strength between the surface sheet and the surface sheet is set to be smaller than that between the wing portions and the surface sheet.

17. An absorbent article according to claim 2, wherein bonding strength between the surface sheet and the liquid-impermeable anti leak sheet is set to be smaller than that between the wing portions and the surface sheet.

18. An absorbent article according to claim 3, wherein bonding strength between the surface sheet and the liquid-impermeable anti leak sheet is set to be smaller than that between the wing portions and the surface sheet.
